(19) United States
(12) Patent Application Publication
(10) Pub. No.: US 2019/0076526 A1
(43) Pub. Date: Mar. 14, 2019

(54) UPCONVERSION NANOPARTICLE, HYALURONIC ACID-UPCONVERSION NANOPARTICLE CONJUGATE, AND A PRODUCTION METHOD THEREOF USING A CALCULATION FROM FIRST PRINCIPLES

(71) Applicants: POSCO, Pohang-si (KR); POSTECH ACADEMY-INDUSTRY FOUNDATION, Pohang-si (KR)

(72) Inventors: Sei Kwang HAHN, Pohang-si (KR); Seulgi HAN, Asan-si (KR); Hyun Woo LEE, Pohang-si (KR); Kyoo KIM, Pohang-si (KR)

(21) Appl. No.: 15/703,194

(22) Filed: Sep. 13, 2017

(51) Int. Cl. A61K 41/00; C09K 11/02; C09K 11/77; A61K 47/69; A61N 5/06

(52) U.S. Cl. CPC A61K 41/008; C09K 11/025; B82Y 40/00; A61K 47/6939; A61N 5/062; C09K 11/7773

(57) ABSTRACT

An upconversion nanoparticle includes at least one host selected from LiYF₄, NaY, NaYF₄, NaGdF₄, and CaF₃, at least one sensitizer selected from Sm³⁺, Nd³⁺, Dy³⁺, Ho³⁺, and Yb³⁺ doped in the at least one host, and at least one activator selected from Er³⁺, Ho³⁺, Tm³⁺, and Eu³⁺ doped in the at least one host. The upconversion nanoparticle is designed using a calculation from first principles to absorb light in the near-infrared wavelength range whose stability is ensured. Further, a hyaluronic acid-upconversion nanoparticle conjugate, in which the upconversion nanoparticle as described above is bonded to hyaluronic acid, is provided to be used in various internal sites with a hyaluronic acid receptor, particularly enables targeting, and increases an internal retention period and biocompatibility thereof.

### TECHNICAL FIELD

The present disclosure relates to an upconversion nanoparticle, a hyaluronic acid-upconversion nanoparticle conjugate, and a production method thereof using a calculation from first principles, and more particularly, to an upconversion nanoparticle, a hyaluronic acid-upconversion nanoparticle conjugate, and a production method thereof using a calculation from first principles that may be designed to absorb light having a near-infrared wavelength, using a calculation from first principles.

### BACKGROUND

Calculation from first principles, as a calculation method based on quantum mechanics, is a method of calculating the properties of a substance without the help of other empirical values except for the positions and types of atoms. Due to such a feature, calculation from first principles may calculate realistic physical quantities to facilitate a direct comparison between the calculated physical quantities and experimental results and to have predictive abilities. Quantum mechanics were established in the 20th century and verified through experimentation. It was known that quantum mechanics could be used to calculate the behavior of electrons using Erwin Schrodinger's wave equation. However, such a method has limitations in describing many electrons interacting with each other within solids, and the practical use of the method is limited to calculating the state of individual atoms or the quantum state of simple molecules consisting of several atoms. In order to describe the state of solids, density functional theory (DFT) describing the behavior of quasiparticles not interacting with each other has primarily been used, in lieu of using various Schrodinger wave equations for electrons interacting with each other. It was verified that such a method could describe the physical properties of many common solids properly, and could have predictive abilities, but could not describe localized electrons correctly.

Information on atomic multiplet energy and on state function is required to replicate energy scale important in energy transfer upconversion (ETU) properly. The distribution feature of atomic multiplet energy levels may be changed according to various structure factors, such as types of atom, electron-electron interaction screening, and a crystal field caused by peripheral ligand atoms. DFT is a proper method to describe such structure factors properly.

Using the calculation from first principles, upconversion nanoparticles, having a nanosize diameter, may be designed, and may be synthesized by doping a host with trivalent lanthanide-based ions, and may be nanomaterials, having the characteristics of emitting light having a short wavelength by absorbing light having a long wavelength, based on an ETU phenomenon between f-f orbitals. Conventional upconversion nanoparticles are based on a mechanism system that uses various lanthanide-based ions as a sensitizer and transfers energy having a triplet or quadruplet energy level to ions doped with various activators, thus upconverting light.

It was known that the long wavelength of the near-infrared wavelength range could be transmitted up to about a 3.5 cm depth, based on a wavelength of 808 nm. The depth increases as the wavelength is increased. However, as body tissues, and water present in blood, absorb light having a long wavelength, an actual depth to which a laser beam is transmitted to skin decreases gradually as the wavelength exceeds 808 nm. However, the longer the wavelength is, the lower skin invasion according the intensity of the laser beam is, and thus stability may be increased. Furthermore, with the permission of the Ministry of Food and Drug Safety, medical equipment companies, such as Lutronic Corporation and others, are developing and producing medical laser equipment, which has neodymiun:yttrium aluminum garnet lasers (Nd:YAG) mounted therein to emit near-infrared light having a wavelength of 1,064 nm, and which is used in the treatment of skin diseases, eye diseases, or the like. Thus, there exists a need for the development of upconversion nanoparticles that may absorb and use near-infrared light having a wavelength of 1,064 nm.

### SUMMARY

An aspect of the present disclosure may provide an upconversion nanoparticle that may be designed to absorb light in the near-infrared wavelength range whose stability is ensured, using a calculation from first principles.

Another aspect of the present disclosure may provide a hyaluronic acid-upconversion nanoparticle conjugate, in which the upconversion nanoparticle may be bonded to hyaluronic acid, so as to be used in various internal sites with a hyaluronic acid receptor, may particularly enable targeting, and may increase an internal retention period and biocompatibility thereof.

According to an aspect of the present disclosure, an upconversion nanoparticle may include: at least one host selected from LiYF₄, NaY, NaYF₄, NaGdF₄, and CaF₃; at least one sensitizer selected from Sm³⁺, Nd³⁺, Dy³⁺, Ho³⁺, and Yb³⁺ doped in the at least one host; and at least one activator selected from Er⁺, Ho³⁺, Tm³⁺, and Eu³⁺ doped in the at least one host.

The upconversion nanoparticle may be determined by calculating an optimal chemical composition of a lanthanide-based ion-doped upconversion nanoparticle absorbing light having at least one wavelength among wavelengths of 808 nm, 980 nm, and 1,064 nm, using a calculation from first principles.

The upconversion nanoparticle may be configured to absorb light having at least one wavelength among wavelengths of 808 nm, 980 nm, and 1,064 nm to emit visible light.

A mole ratio of the at least one sensitizer to the at least one host may be 80:10 to 80:60.

According to another aspect of the present disclosure, a hyaluronic acid-upconversion nanoparticle conjugate may include: the upconversion nanoparticle; and hyaluronic acid bonded to the upconversion nanoparticle, or a derivative of hyaluronic acid.

The hyaluronic acid-upconversion nanoparticle conjugate may further include a photosensitizer.

The photosensitizer may include at least one selected from chlorine e6 (Ce6), a porphyrin-based photosensitizer, and a non-porphyrin-based photosensitizer.

1 to 2 parts by weight of the photosensitizer may be bonded to 1 part by weight of the upconversion nanoparticle.

The derivative of hyaluronic acid may be hyaluronic acid substituted with cystamine, having a structure represented by the following Chemical Formula 1,

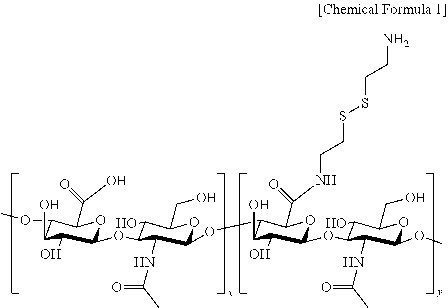

where x and y are integers selected from 16 to 2,500, respectively.

The cystamine may be substituted at a replacement ratio of 10% to 21% with respect to the hyaluronic acid.

A weight ratio of the upconversion nanoparticle to the hyaluronic acid or the derivative of hyaluronic acid may be 1:1 to 4:1.

According to another aspect of the present disclosure, a method of producing an upconversion nanoparticle may include:

(a) producing a solution by mixing a host precursor, a sensitizer, an activator, and a solvent; and (b) producing an upconversion nanoparticle by subjecting the solution to a heat treatment.

The host precursor may include at least one selected from YCl₃.H₂O, YbCl₃.H₂O, SmCl₃.H₂O, NdCl₃.H₂O, GdCl₃.H₂O, Ca(CF₃COO)₂, CF₃COONa, Y(CF₃COO)₃, Yb(CF₃COO)₃, Gd(CF₃COO)₃, Sm(CF₃COO)₃, Nd(CF₃COO)₃, NH₄F, and NaOH.

The solvent may include octadecene-1.

The solution may further include at least one selected from oleic acid and oleylamine.

The heat treatment may be conducted at 250° C. to 400° C.

According to another aspect of the present disclosure, a method of producing a hyaluronic acid-upconversion nanoparticle conjugate may include: (a) bonding the upconversion nanoparticle to hyaluronic acid or a derivative of hyaluronic acid.

The bonding may include (a′) mixing or dissolving the hyaluronic acid or the derivative of hyaluronic acid with the upconversion nanoparticle, and then adding, as a catalyst, 1-(3-dimethylaminopropyl)-3-ethylcarbodiimide hydrochloride (EDC) to a mixture or a solution, so as to react the mixture or the solution with the EDC.

The method of producing a hyaluronic acid-upconversion nanoparticle conjugate may further include (a-1) modifying a surface of the upconversion nanoparticle, prior to operation (a′).

The surface of the upconversion nanoparticle may be modified using at least one selected from polyallylamine, polymethylmethacrylate (PMMA), 3-aminopropyltriethoxysilane (APTES), tetraethyl orthosilicate (TEOS), 3,4-dihydroxyphenylalanine (DOPA), and cetyltrimethylammoniumbromide (CTAB).

According to another aspect of the present disclosure, a composition for optogenetics applicable to optogenetics may include the hyaluronic acid-upconversion nanoparticle conjugate as an active ingredient.

The composition for optogenetics may be configured to be used to control nerve cells, using a laser beam having at least one wavelength among wavelengths of 808 nm, 980 nm, and 1,064 nm.

According to another aspect of the present disclosure, a composition for photodynamic therapy may include the hyaluronic acid-upconversion nanoparticle conjugate as an active ingredient.

The composition for photodynamic therapy may be configured to be used in the treatment of skin diseases or cancers.

The composition for photodynamic therapy may be configured as a patch preparation, a depot preparation, or an external preparation.

According to another aspect of the present disclosure, a non-invasive internal light source delivery system using transdermal delivery of the hyaluronic acid-upconversion nanoparticle conjugate may be provided.

The non-invasive internal light source delivery system may be configured to be used in the treatment and diagnosis of cancers, skin diseases, or eye diseases.

The non-invasive internal light source delivery system may be configured to be used in fluorescent tattoos.

The non-invasive internal light source delivery system may be configured to be applicable to cell therapy, using a hydrogel produced through a physical host-guest reaction between a hyaluronic acid-cucurbituril conjugate, in which cucurbituril[6] may be bonded to hyaluronic acid substituted with cystamine, and/or a Ce6-hyaluronic acid-cucurbituril conjugate, in which Ce6 may be additionally bonded to the hyaluronic acid-cucurbituril conjugate as a photosensitizer, and a hyaluronic acid-upconversion nanoparticle conjugate.

### BRIEF DESCRIPTION OF DRAWINGS

The above and other aspects, features, and advantages of the present disclosure will be more clearly understood from the following detailed description, taken in conjunction with the accompanying drawings, in which:

FIGS. 1A and 1B are schematic views of structures of an upconversion nanoparticle, a hyaluronic acid-upconversion nanoparticle conjugate, and a hyaluronic acid-upconversion nanoparticle-photosensitizer conjugate, further including a photosensitizer, and production methods thereof;

FIG. 2 illustrates changes in atomic multiplet energy of the upconversion nanoparticle (LiYF₄ doped with Sm³⁺) produced according to Exemplary embodiment 1;

FIG. 3 illustrates changes in atomic multiplet energy of a upconversion nanoparticle produced according to Exemplary embodiment 2 and of an upconversion nanoparticle (CaF₃ doped with Er³⁺);

FIGS. 4A and 4B are a transdermal delivery process of the hyaluronic acid-upconversion nanoparticle conjugate produced according to Exemplary embodiment 2, and a result of observing fluorescence of the hyaluronic acid-upconversion nanoparticle conjugate delivered in vivo to the abdomen of a laboratory mouse;

FIGS. 5A through 5C are results of analyzing the upconversion nanoparticle produced according to Exemplary embodiment 2, a silica-coated upconversion nanoparticle produced in a manufacturing process of Exemplary embodiment 4, and a hyaluronic acid-upconversion nanoparticle conjugate produced according to Exemplary embodiment 4 through a transmission electron microscope (TEM);

FIGS. 6A and 6B are results of measuring changes in fluorescent intensity of the upconversion nanoparticle produced according to Exemplary embodiment 2, and of fluorescent efficiency thereof;

FIG. 7 is a result of measuring cytotoxicity of the hyaluronic acid-upconversion nanoparticle conjugate produced according to Exemplary embodiment 4 and upconversion nanoparticle-polyallylamine whose surface is coated with polyallylamine before bonding hyaluronic acid to an upconversion nanoparticle according to Comparative Example 1 through the MTT assay; and

FIG. 8 is a result of transdermally delivering the hyaluronic acid-upconversion nanoparticle conjugate produced according to Exemplary embodiment 4 and distilled water to the abdomen of a laboratory mouse, radiating a laser beam, and observing the hyaluronic acid-upconversion nanoparticle conjugate through a two-photon microscope.

### DETAILED DESCRIPTION

Hereinafter, exemplary embodiments in the present disclosure are described in detail with reference to the accompanying drawings in order for those skilled in the art to be able to readily practice them.

However, the following description is not intended to limit the present disclosure to specific embodiments. Also, while describing the aspects, detailed descriptions about related well-known functions or configurations that may depart from the gist of the present disclosure will be omitted.

The terminology provided herein is merely used for the purpose of describing particular embodiments, and is not intended to limit the exemplary embodiments in the present disclosure. The singular forms “a,” “an” and “the” are intended to include the plural forms as well, unless the context clearly indicates otherwise. It should be understood that the terms “comprises,” “comprising,” “includes,” and/or “including,” when used herein, specify the presence of stated features, integers, steps, operations, elements, components and/or combinations thereof, but do not preclude the presence or addition of one or more other features, integers, steps, operations, elements, components and/or combinations thereof.

An upconversion nanoparticle, according to an exemplary embodiment, is described hereinafter in detail. This is presented as an example, not intended to limit the exemplary embodiments in the present disclosure, and only defined by the scope of claims to be described later.

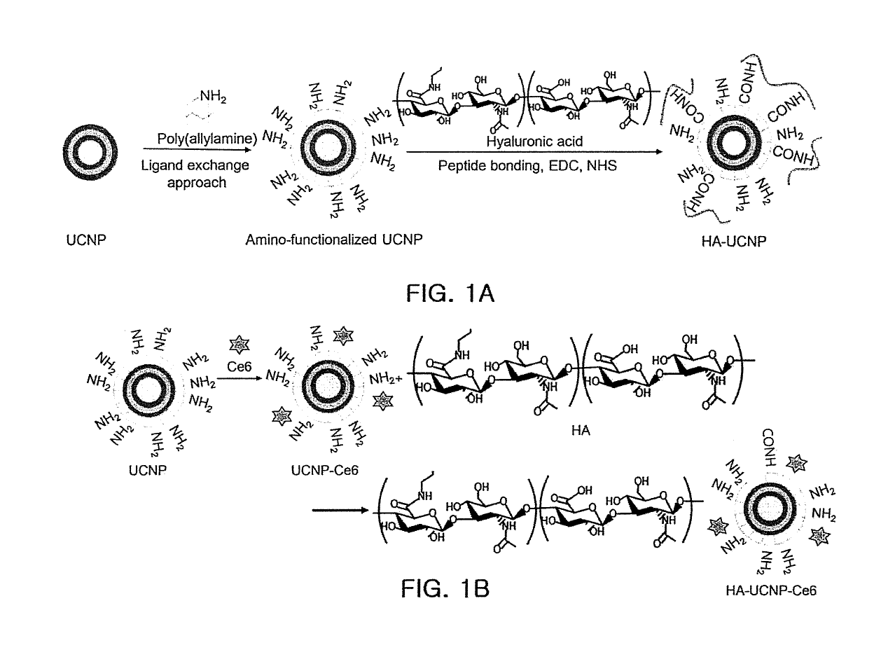

FIGS. 1A and 1B are schematic views illustrating structures of an upconversion nanoparticle UCNP, a hyaluronic acid-upconversion nanoparticle conjugate HA-UCNP, and a hyaluronic acid-upconversion nanoparticle-photosensitizer conjugate HA-UCNP-Ce6, further including a photosensitizer, and production methods thereof. Here, hyaluronic acid, photosensitizer Ce6, modifier poly(allylamine), and the like are mentioned. However, the present disclosure is not limited thereto.

The upconversion nanoparticle, according to an exemplary embodiment, may include: at least one host selected from LiYF₄, NaY, NaYF₄, NaGdF₄, and CaF₃; at least one sensitizer selected from Sm³⁺, Nd³⁺, Dy³⁺, Ho³⁺, and Yb³⁺ doped in the at least one host; and at least one activator selected from Er³⁺, Ho³⁺, Tm³⁺, and Eu³⁺ doped in the at least one host.

The upconversion nanoparticle may increase efficiency of an upconversion nanoparticle, according to the related art, that may absorb light having wavelengths of 808 nm and 980 nm, and may be determined by calculating an optimal chemical composition of a novel lanthanide-based ion-doped upconversion nanoparticle that may absorb light having a wavelength of 1,064 nm, using a calculation from first principles. The at least one host and the at least one sensitizer may be determined by predicting multiplet energy levels of various lanthanide-based ions, using a calculation from first principles. For example, Sm³⁺ ions may be derived as a sensitizer, having significantly increased efficiency and absorbing a wavelength of 1,064 nm. In detail, multiplet energy levels of doped ions, such as Sm³⁺, Dy³⁺, and Ho³⁺, due to an interaction with the at least one host of the upconversion nanoparticle may be calculated using first principles. The upconversion material may be theoretically designed and experimentally synthesized using a method of obtaining structural information on trivalent lanthanide-based ions doped in the at least one host using density functional theory (DFT), and of obtaining an absorption and emission spectrum by calculating atomic multiplet energy of the lanthanide-based ions and transition thereof between atomic multiplet energy levels thereof using variables extracted from the structural information.

Hamiltonian as represented by the following Formula 1 may be diagonalized to precisely calculate the atomic multiplet energy levels.

H_{f}=Hₑₗ₋ₑₗ+H_{SOC}+H_{CEF}

Hₑₗ₋ₑₗ=Σ₁Σₘ_{₁}₋ₘ_{₂}Σ_{σ}_{₁}_{σ}_{₂}fₘ_{₁}ₘ_{₂}ₘ_{₃}ₘ_{₄}fₘ_{₁}_{σ}_{₁}⁺fₘ_{₂}_{σ}_{₂}⁺fₘ_{₁}_{σ}_{₁}fₘ_{₄}_{σ}_{₄}

H_{SOC}=Σ₁Σ_{mm′}Σ_{σσ′}λ_{SOC}C_{mσ′σ′}f_{mσ}⁺f_{m′σ′}

H_{CEF}=Σ_{f}Σ_{mm′}Σ_{σ}A_{mm′}f_{mσ}⁺f_{m′σ} [Formula 1]

Hₑₗ₋ₑₗ is a term relating to an electron-electron interaction, H_{soc} is a term relating to a spin-orbit interaction, and H_{CEF} is a term relating to a crystal field.

An absorption spectrum may be determined by transition of the lanthanide-based ions between the atomic multiplet energy levels, and the distribution of the atomic multiplet energy levels may be dependent on a type of atom of the at least one sensitizer. It may be found, through an experiment according to the related art and a calculation of the atomic multiplet energy, that the Sm³⁺ ions have energy levels that are able to absorb near-infrared light having a wavelength of 1,064 nm. Further, the distribution of the atomic multiplet energy levels may be dependent on a crystal field, varying according to a type of host and to a position of a doped atom.

Thus, the Sm³⁺ ions, having significantly increased absorption intensity, among the lanthanide-based ions having energy levels that absorb near-infrared light having a wavelength of 1,064 nm, whose stability is verified, may be selected, and components of the upconversion nanoparticle may be designed.

The upconversion nanoparticle may absorb light having at least one wavelength among wavelengths of 808 nm, 980 nm, and 1,064 nm to emit visible light.

A mole ratio of the at least one sensitizer to the at least one host may be 80:10 to 80:60, preferably 80:10 to 80:30, and more preferably 80:18 to 80:25.

The hyaluronic acid-upconversion nanoparticle conjugate, according to an exemplary embodiment, is described hereinafter.

In the present specification, bonding may be chemical or physical bonding, preferably chemical bonding, specifically covalent bonding, ionic bonding, or coordinate bonding, and preferably covalent bonding.

The hyaluronic acid-upconversion nanoparticle conjugate, according to an exemplary embodiment, may include the upconversion nanoparticle, and hyaluronic acid bonded to the upconversion nanoparticle or a derivative thereof.

The upconversion nanoparticle may be used in various internal sites in which a hyaluronic acid receptor is present by allowing the hyaluronic acid, a supermolecule having biocompatibility, to be interposed between portions of a surface of the upconversion nanoparticle. In particular, the upconversion nanoparticle may enable selective targeting of sites below the skin or in the eyes in which a large amount of hyaluronic acid receptors are present, and may increase an internal retention period and biocompatibility thereof.

For example, a weight average molecular weight of the hyaluronic acid may range from 10,000 to 1,000,000, but a molecular weight of the hyaluronic acid available in an exemplary embodiment is not limited thereto. When the molecular weight of the hyaluronic acid is equal to or less than 10,000, the ability of the hyaluronic acid to maintain physiological stability of the upconversion nanoparticle may be decreased. When the molecular weight of the hyaluronic acid is equal to or greater than 1,000,000, the total size of the upconversion nanoparticle may grow to be significantly larger.

The hyaluronic acid-upconversion nanoparticle conjugate may further include a photosensitizer.

The photosensitizer may be at least one selected from chlorine e6 (Ce6), a porphyrin-based photosensitizer, and a non-porphyrin-based photosensitizer, preferably chlorine e6.

1 to 3 parts by weight of the photosensitizer, preferably 1 to 2 parts by weight thereof, and more preferably 2 parts by weight thereof may be bonded to 1 part by weight of the upconversion nanoparticle.

When a functional group of the porphyrin-based photosensitizer is carboxylic acid, the porphyrin-based photosensitizer may react with an amino group of the upconversion nanoparticle to create an amide bond between the carboxylic acid and the amino group. Otherwise, the upconversion nanoparticle may form a micelle, include the porphyrin-based photosensitizer in the micelle, and deliver the micelle in vivo.

The derivative of hyaluronic acid may be hyaluronic acid substituted with cystamine, having a structure represented by the following Chemical Formula 1,

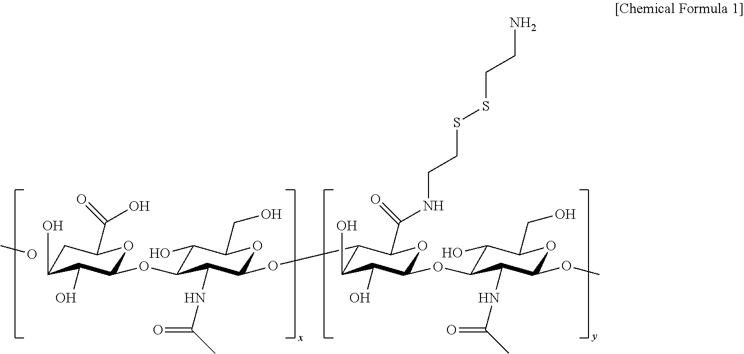

where x and y are integers selected from 16 to 2,500, respectively.

Further, x and y may be determined according to replacement ratios. For example, when the replacement ratios are 30%, 20%, and 10%, respectively, x and y may be integers present at a ratio of 7:3, 8:2, or 9:1, respectively.

The cystamine may be substituted at a replacement ratio of 10% to 21% with respect to the hyaluronic acid, preferably 12% to 19%, and more preferably 14% to 16%.

A weight ratio of the upconversion nanoparticle to the hyaluronic acid or the derivative of hyaluronic acid may be 1:1 to 4:1, preferably 2:1 to 4:1, and more preferably 3:1 to 4:1.

A method of producing an upconversion nanoparticle, according to an exemplary embodiment, is described hereinafter.

First, a solution may be produced by mixing a host precursor, a sensitizer, an activator, and a solvent (operation 1).

The host precursor may include at least one selected from YCl₃.H₂O, YbCl₃.H₂O, SmCl₃.H₂O, NdCl₃.H₂O, GdCl₃.H₂O, Ca(CF₃COO)₂, CF₃COONa, Y(CF₃COO)₃, Yb(CF₃COO)₃, Gd(CF₃COO)₃, Sm(CF₃COO)₃, Nd(CF₃COO)₃, NH₄F, and NaOH.

For example, the solvent may be octadecene-1.

The solution may further include oleic acid, oleylamine, or the like, preferably oleic acid. The oleic acid may prevent aggregation, while serving as a passivating ligand.

Subsequently, an upconversion nanoparticle may be produced by subjecting the solution to a heat treatment (operation 2).

The heat treatment may be conducted at 250° C. to 400° C., preferably 280° C. to 350° C., and more preferably 290° C. to 330° C.

A method of producing a hyaluronic acid-upconversion nanoparticle conjugate, according to an exemplary embodiment, is described hereinafter.

First, the upconversion nanoparticle may be bonded to hyaluronic acid or a derivative of hyaluronic acid (operation a).

The bonding may include mixing or dissolving the hyaluronic acid or the derivative of hyaluronic acid with the upconversion nanoparticle, and then adding, as a catalyst, 1-(3-dimethylaminopropyl)-3-ethylcarbodiimide hydrochloride (EDC) to a mixture or a solution, so as to react the mixture or the solution with the EDC (operation a′).

The method of producing a hyaluronic acid-upconversion nanoparticle conjugate may further include modifying a surface of the upconversion nanoparticle, prior to operation a′ (operation a-1).

The surface of the upconversion nanoparticle may be modified using at least one selected from polyallylamine, polymethylmethacrylate (PMMA), 3-aminopropyltriethoxysilane (APTES), tetraethyl orthosilicate (TEOS), 3,4-dihydroxyphenylalanine (DOPA), and cetyltrimethylammoniumbromide (CTAB).

The method of producing a hyaluronic acid-upconversion nanoparticle conjugate may further include removing the EDC, subsequent to operation a′.

Various applications of the hyaluronic acid-upconversion nanoparticle conjugate, according to an exemplary embodiment, are described hereinafter.

A composition for optogenetics applicable to optogenetics including the hyaluronic acid-upconversion nanoparticle conjugate as an active ingredient may be provided.

The composition for optogenetics may be used to control nerve cells, using a laser beam having at least one wavelength among wavelengths of 808 nm, 980 nm, and 1,064 nm.

A composition for photodynamic therapy including the hyaluronic acid-upconversion nanoparticle conjugate as an active ingredient may be provided.

The composition for photodynamic therapy may be used in the treatment of skin diseases or cancers.

The composition for photodynamic therapy may be a patch preparation, a depot preparation, or an external preparation.

A non-invasive internal light source delivery system using transdermal delivery of the hyaluronic acid-upconversion nanoparticle conjugate may be provided.

The non-invasive internal light source delivery system may be used in the treatment and diagnosis of cancers, skin diseases, or eye diseases.

The non-invasive internal light source delivery system may be used in fluorescent tattoos.

The non-invasive internal light source delivery system may be configured to be applicable to cell therapy, using a hydrogel produced through a physical host-guest reaction between a hyaluronic acid-cucurbituril conjugate, in which cucurbituril[6] may be bonded to hyaluronic acid substituted with cystamine, and/or a Ce6-hyaluronic acid-cucurbituril conjugate, in which Ce6 may be additionally bonded to the hyaluronic acid-cucurbituril conjugate as a photosensitizer, and a hyaluronic acid-upconversion nanoparticle conjugate.

### Exemplary Embodiment

Exemplary embodiments are described hereinafter. However, such exemplary embodiments are provided as examples, and the scope of the present disclosure is not limited thereto.

### Exemplary Embodiment 1: Production of Upconversion Nanoparticle

A mixed solution was produced by adding SmCl₃.H₂O, YCl₃.H₂O, YbCl₃.H₂O, NH₄F, and NaOH to a solvent, containing 15 ml of octadecene-1 and 6 ml of oleic acid, in an inert gas atmosphere. The mixed solution was reacted for 30 minutes at 150° C., subjected to a closed environment using nitrogen (N), and thermally treated at 315° C. for one and a half hours. Subsequently, the temperature was adjusted to room temperature, and ethanol was added to the mixed solution to terminate the reaction. Thus, an upconversion nanoparticle was produced. The upconversion nanoparticle was separated using a centrifuge.

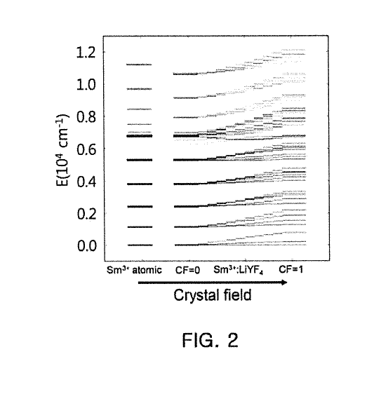

Changes in atomic multiplet energy of the upconversion nanoparticle (LiYF₄ doped with Sm³⁺) are illustrated in FIG. 2.

### Exemplary Embodiment 2: Production of Upconversion Nanoparticle

An upconversion nanoparticle was produced in the same manner as Exemplary Embodiment 1, except that ErCl₃.H₂O was used in place of SmCl₃.H₂O.

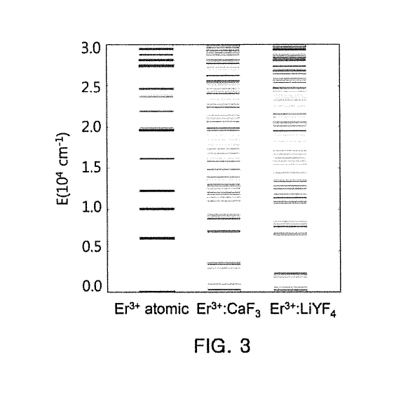

Illustrated in FIG. 3 are changes in atomic multiplet energy of the upconversion nanoparticle and an upconversion nanoparticle, in which CaF₃ was doped with Er³⁺.

### Exemplary Embodiment 3: Surface Coating of Upconversion Nanoparticle

The upconversion nanoparticle produced according to Exemplary Embodiment 1 was dissolved in cyclohexane, and ethanol containing a polyallylamine aqueous solution (20 wt %, about M.W. 17,000) dissolved therein was added to a solution to substitute oleic acid present on a surface of the upconversion nanoparticle with polyallylamine.

The oleic acid present on the surface of the upconversion nanoparticle was substituted with APTES by performing, on the upconversion nanoparticle produced according to Exemplary Embodiment 1, a water-in-oil reverse method using APTES and TEOS, and then the upconversion nanoparticle was coated with 10 nm thickness silica by injecting TEOS thereinto at a rate of 1 ml/h, using a syringe pump.

### Exemplary Embodiment 4: Production of Hyaluronic Acid-Upconversion Nanoparticle Conjugate

The upconversion nanoparticle surface-coated with the silica or the polyallylamine and produced according to Exemplary Embodiment 3, and hyaluronic acid were dissolved in distilled water, and then EDC was added to a solution as a catalyst, so as to react the solution with the EDC. Thus, a hyaluronic acid-upconversion nanoparticle conjugate was produced.

### Exemplary Embodiment 5: Production of Hyaluronic Acid-Upconversion Nanoparticle Conjugate

A hyaluronic acid-upconversion nanoparticle conjugate was produced in the same manner as Exemplary Embodiment 3, except that the upconversion nanoparticle produced according to Exemplary Embodiment 2 was used, in lieu of the upconversion nanoparticle produced according to Exemplary Embodiment 1.

### Exemplary Embodiment 6: Production of Hyaluronic Acid-Upconversion Nanoparticle-Photosensitizer Conjugate

The hyaluronic acid-upconversion nanoparticle conjugate produced according to Exemplary Embodiment 4, and chlorine e6 (Ce6), a photosensitizer, were dissolved in distilled water, and then EDC was added to a solution as a catalyst, so as to react the solution with the EDC. Thus, a hyaluronic acid-upconversion nanoparticle-photosensitizer conjugate was produced.

### Exemplary Embodiment 7: Production of Hyaluronic Acid-Upconversion Nanoparticle-Photosensitizer Conjugate

A hyaluronic acid-upconversion nanoparticle-photosensitizer conjugate was produced in the same manner as Exemplary Embodiment 6, except that the hyaluronic acid-upconversion nanoparticle conjugate produced according to Exemplary Embodiment 5 was used, in lieu of the hyaluronic acid-upconversion nanoparticle conjugate produced according to Exemplary Embodiment 4.

### Comparative Exemplary Embodiment 1: Production of Hyaluronic Acid-Organic Carbon Dot Conjugate

A mixed solution was produced by mixing 15 ml of octadecene-1 with 1.5 g of hexadecylamine-1, and heated to a high temperature of 300° C. in an argon (Ar) environment. 1 g of citric acid was added to the mixed solution, and then reacted for three hours to produce an organic carbon dot. The organic carbon dot and a hyaluronic acid-tetrabutylammonium (TBA) derivative were dissolved in a dimethyl sulfoxide (DMSO) solvent at a ratio of 4 parts by weight of the organic carbon dot to 1 part by weight of the hyaluronic acid to be mixed with each other, and were reacted at 37° C. overnight, using (benzotriazol-1-yloxy)tris(dimethylamino)phosphonium hexafluorophosphate (BOP) and N,N-Diisopropylethylamine (DIPEA) catalysts. Subsequent to the termination of the reaction, a product was refined through dialysis, and a hyaluronic acid-organic carbon dot conjugate was produced using a freeze-drying method.

### Experimental Exemplary Embodiment

### Experimental Exemplary Embodiment 1: Confirmation of Transdermal Delivery

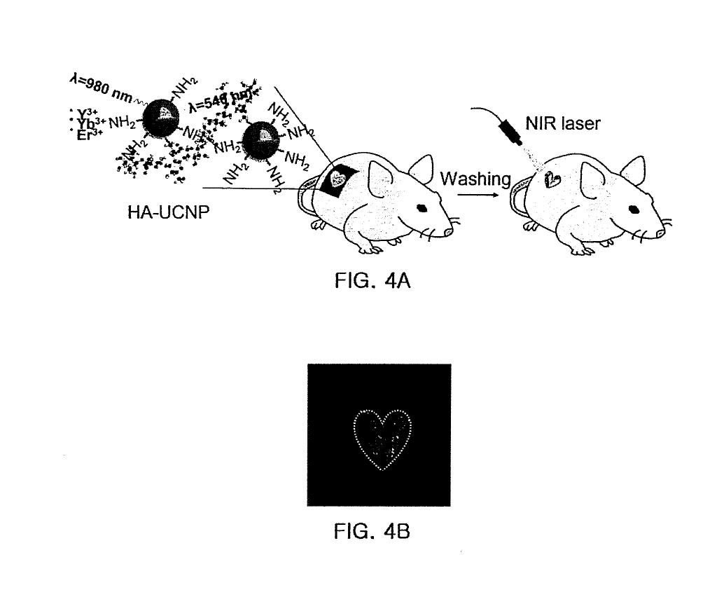

Transdermal delivery of the hyaluronic acid-upconversion nanoparticle conjugate (HA-UCNP) produced according to Exemplary Embodiment 4 is illustrated in FIG. 4A. Illustrated in FIG. 4B is a result of delivering, in vivo, the hyaluronic acid-upconversion nanoparticle conjugate, produced according to Exemplary Embodiment 4, to the abdomen of a laboratory mouse in an amount of 0.625 mg per 1 kg of body weight of the laboratory mouse in various patterns at an aqueous solution concentration of 125 μg/ml and observing fluorescence of the hyaluronic acid-upconversion nanoparticle conjugate.

Referring to FIGS. 4A and 4B, it can be seen that the hyaluronic acid-upconversion nanoparticle conjugate produced according to Exemplary Embodiment 4 is delivered particularly deeply into the skin of the laboratory mouse.

Thus, it may be determined that an upconversion nanoparticle may be utilized in treatment and diagnosis using light by being delivered particularly deeply into skin, using a large amount of hyaluronic acid receptors present in the skin.

### Experimental Exemplary Embodiment 2: TEM Analysis

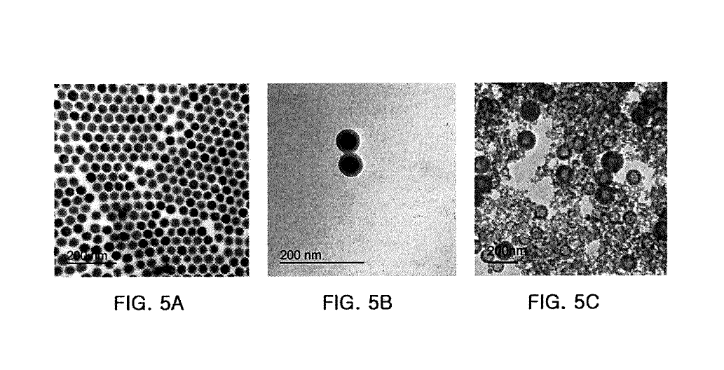

Illustrated in FIGS. 5A through 5C are results of analyzing the upconversion nanoparticle (FIG. 5A) produced according to Exemplary Embodiment 1, the silica-coated upconversion nanoparticle (FIG. 5B) produced in the manufacturing process of Exemplary embodiment 4, and the hyaluronic acid-upconversion nanoparticle conjugate (FIG. 5C) produced according to Exemplary embodiment 4 through a TEM.

Referring to FIGS. 5A through 5C, it can be seen that the upconversion nanoparticle produced according to Exemplary Embodiment 1 is uniformly synthesized to have a nanosize of 30 nm to 40 nm. Further, it can be seen that the silica-coated upconversion nanoparticle produced in the manufacturing process of Exemplary embodiment 4 is uniformity coated with 10 nm thickness silica. It can be seen that the hyaluronic acid-upconversion nanoparticle conjugate produced according to Exemplary embodiment 4 contains the hyaluronic acid, covering a periphery of the upconversion nanoparticle.

### Experimental Exemplary Embodiment 3: Analysis of Fluorescence Intensity and Efficiency

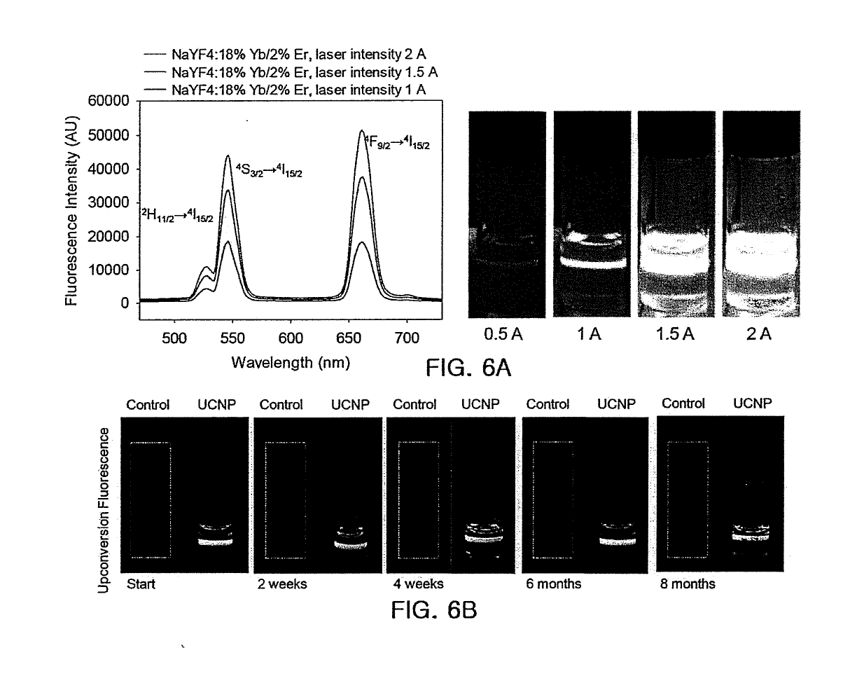

Illustrated in FIG. 6A are changes in fluorescence intensity of the upconversion nanoparticle (NaYF₄:18% Yb/2% Er), produced according to Exemplary Embodiment 1, according to laser beam intensity. Illustrated in FIG. 6B are a result of measuring fluorescence efficiency for a period of eight months, subsequent to the synthesis.

Referring to FIGS. 6A and 6B, it can be seen that, as the laser beam intensity increases, intensity of red light having a wavelength of 670 nm from the upconversion nanoparticle produced according to Exemplary Embodiment 1 increases. Further, it can be seen that fluorescence intensity of the upconversion nanoparticle produced according to Exemplary Embodiment 1 is maintained for a period of eight months, subsequent to the synthesis.

### Experimental Exemplary Embodiment 4: Confirmation of Cytotoxicity

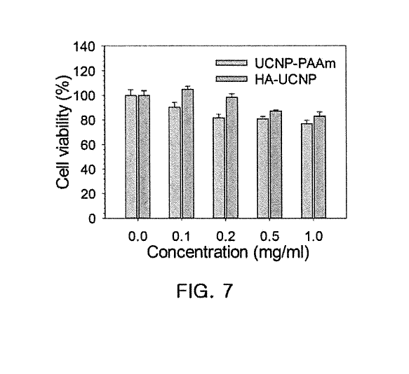

Illustrated in FIG. 7 is a result of targeting the hyaluronic acid-upconversion nanoparticle conjugate (HA-UCNP) produced according to Exemplary Embodiment 4 and the upconversion nanoparticle produced according to Comparative Exemplary Embodiment 1 and having the polyallylamine interposed between the portions of the surface of the upconversion nanoparticle to an NIH3T3 cell, a skin cell, incubating the NIH3T3 cell for 24 hours, and measuring cytotoxicity through MTT assay.

The respective hyaluronic acid-upconversion nanoparticle conjugates were tested with aqueous solutions, having 0, 0.1, 0.2, 0.5, and 1.0 mg/ml concentrations.

Referring to FIG. 7, a cell survival rate of 80% or more may be confirmed from an aqueous solution, having a high concentration of 1 mg/ml, of the hyaluronic acid-upconversion nanoparticle conjugate produced according to Exemplary Embodiment 4.

### Experimental Exemplary Embodiment 5: Analysis of Transdermal Delivery

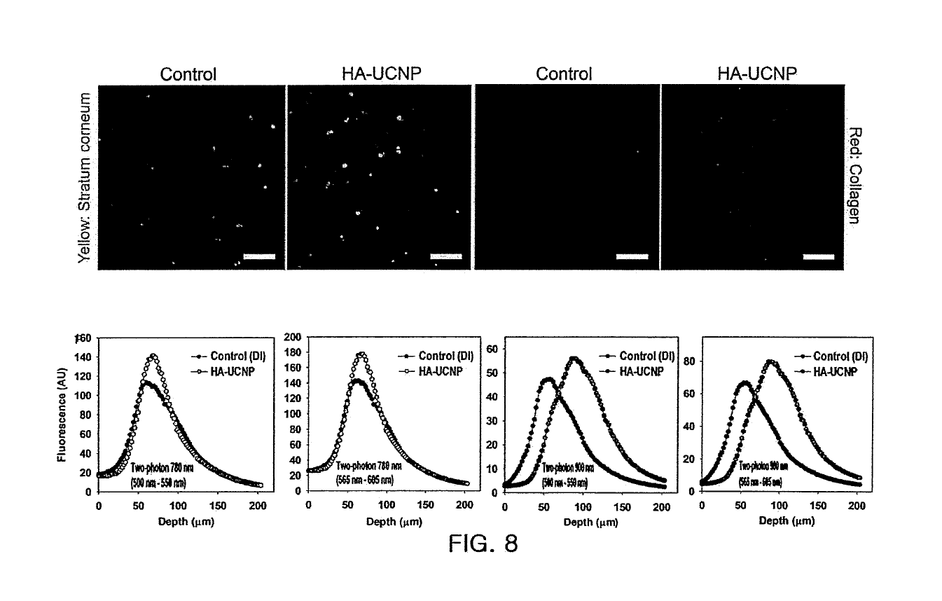

Illustrated in FIG. 8 is a result of transdermally delivering the hyaluronic acid-upconversion nanoparticle conjugate (HA-UCNP) produced according to Exemplary Embodiment 4 and distilled water (control) to the abdomen of a shaved BALE/c mouse for 30 minutes, radiating a laser beam having a wavelength of 980 nm, and observing the hyaluronic acid-upconversion nanoparticle conjugate with a two-photon microscope.

The hyaluronic acid-upconversion nanoparticle conjugate produced according to Exemplary Embodiment 4 was injected in the form of an aqueous solution, having a concentration of 100 μg/ml.

Referring to FIG. 8, it can be seen that the hyaluronic acid-upconversion nanoparticle conjugate produced according to Exemplary Embodiment 4 is delivered to a collagen layer.

As set forth above, according to an exemplary embodiment, an upconversion nanoparticle may be designed using a calculation from first principles to absorb light in the near-infrared wavelength range whose stability is ensured.

Further, a hyaluronic acid-upconversion nanoparticle conjugate, in which the upconversion nanoparticle may be bonded to hyaluronic acid, may be provided, so as to be used in various internal sites with a hyaluronic acid receptor, may particularly enable targeting, and may increase an internal retention period and biocompatibility thereof.

While exemplary embodiments have been shown and described above, it will be apparent to those skilled in the art that modifications and variations could be made without departing from the scope of the present disclosure, as defined by the appended claims.

What is claimed is:

1. An upconversion nanoparticle, comprising:
at least one host selected from LiYF₄, NaY, NaYF₄, NaGdF₄, and CaF₃;
at least one sensitizer selected from Sm³⁺, Nd³⁺, Dy³⁺, Ho³⁺, and Yb³⁺ doped in the at least one host; and
at least one activator selected from Er³⁺, Ho³⁺, Tm³⁺, and Eu³⁺ doped in the at least one host.

2. The upconversion nanoparticle of claim 1, determined by calculating an optimal chemical composition of a lanthanide-based ion-doped upconversion nanoparticle absorbing light having at least one wavelength among wavelengths of 808 nm, 980 nm, and 1,064 nm, using a calculation from first principles.

3. The upconversion nanoparticle of claim 1, configured to absorb light having at least one wavelength among wavelengths of 808 nm, 980 nm, and 1,064 nm to emit visible light.

4. The upconversion nanoparticle of claim 1, wherein a mole ratio of the at least one sensitizer to the at least one host is 80:10 to 80:60.

5. A hyaluronic acid-upconversion nanoparticle conjugate comprising:
the upconversion nanoparticle according to claim 1; and hyaluronic acid or a derivative of hyaluronic acid bonded to the upconversion nanoparticle.

6. The hyaluronic acid-upconversion nanoparticle conjugate of claim 5, further comprising: a photosensitizer.

7. The hyaluronic acid-upconversion nanoparticle conjugate of claim 6, wherein the photosensitizer is at least one selected from chlorine e6 (Ce6), a porphyrin-based photosensitizer, and a non-porphyrin-based photosensitizer.

8. The hyaluronic acid-upconversion nanoparticle conjugate of claim 7, wherein 1 to 3 parts by weight of the photosensitizer is bonded to 1 part by weight of the upconversion nanoparticle.

9. The hyaluronic acid-upconversion nanoparticle conjugate of claim 5, wherein the derivative of hyaluronic acid is hyaluronic acid substituted with cystamine, having a structure represented by the following Chemical Formula 1, 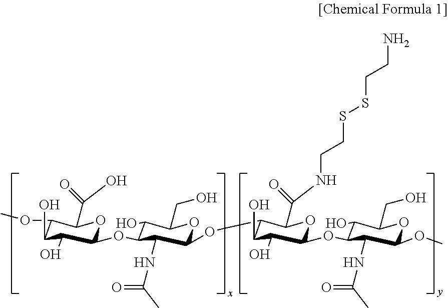
where x and y are integers selected from 16 to 2,500, respectively.

10. The hyaluronic acid-upconversion nanoparticle conjugate of claim 9, wherein the cystamine is substituted at a replacement ratio of 10% to 21% with respect to the hyaluronic acid.

11. The hyaluronic acid-upconversion nanoparticle conjugate of claim 5, wherein a weight ratio of the upconversion nanoparticle to the hyaluronic acid or the derivative of hyaluronic acid is 1:1 to 4:1.

12. A method of producing an upconversion nanoparticle, the method comprising:
(a) producing a solution by mixing a host precursor, a sensitizer, an activator, and a solvent; and
(b) producing an upconversion nanoparticle by subjecting the solution to a heat treatment.

13. The method of claim 12, wherein the host precursor comprises at least one selected from YCl₃.H₂O, YbCl₃.H₂O, SmCl₃.H₂O, NdCl₃.H₂O, GdCl₃.H₂O, Ca(CF₃COO)₂, CF₃COONa, Y(CF₃COO)₃, Yb(CF₃COO)₃, Gd(CF₃COO)₃, Sm(CF₃COO)₃, Nd(CF₃COO)₃, NH₄F, and NaOH.

14. The method of claim 13, wherein the solvent comprises octadecene-1.

15. The method of claim 14, wherein the solution further comprises at least one selected from oleic acid and oleylamine.

16. The method of claim 12, wherein the heat treatment is conducted at 250° C. to 400° C.

17. A method of producing a hyaluronic acid-upconversion nanoparticle conjugate, the method comprising:
(a) bonding the upconversion nanoparticle produced according to claim 12 to hyaluronic acid or a derivative of hyaluronic acid.

18. The method of claim 17, wherein the bonding comprises (a′) mixing or dissolving the hyaluronic acid or the derivative of hyaluronic acid with the upconversion nanoparticle, and then adding, as a catalyst, 1-(3-dimethylaminopropyl)-3-ethylcarbodiimide hydrochloride (EDC) to a mixture or a solution, so as to react the mixture or the solution with the EDC.

19. The method of claim 18, further comprising: (a-1) modifying a surface of the upconversion nanoparticle, prior to operation (a′).

20. The method of claim 19, wherein the surface of the upconversion nanoparticle is modified using at least one selected from polyallylamine, polymethylmethacrylate (PMMA), 3-aminopropyltriethoxysilane (APTES), tetraethyl orthosilicate (TEOS), 3,4-dihydroxyphenylalanine (DOPA), and cetyltrimethylammoniumbromide (CTAB).

21. A composition for optogenetics applicable to optogenetics, the composition for optogenetics comprising:
the hyaluronic acid-upconversion nanoparticle conjugate according to claim 5 as an active ingredient.

22. The composition for optogenetics of claim 21, configured to be used to control nerve cells, using a laser beam having at least one wavelength among wavelengths of 808 nm, 980 nm, and 1,064 nm.

23. A composition for photodynamic therapy, comprising:
the hyaluronic acid-upconversion nanoparticle conjugate according to claim 5 as an active ingredient.

24. The composition for photodynamic therapy of claim 23, configured to be used in the treatment of skin diseases or cancers.

25. The composition for photodynamic therapy of claim 24, configured as a patch preparation, a depot preparation, or an external preparation.

26. A non-invasive internal light source delivery system, configured to use transdermal delivery of the hyaluronic acid-upconversion nanoparticle conjugate according to claim 5.

27. The non-invasive internal light source delivery system of claim 26, configured to be used in the treatment and diagnosis of cancers, skin diseases, or eye diseases.

28. The non-invasive internal light source delivery system of claim 27, configured to be used in fluorescent tattoos.

29. The non-invasive internal light source delivery system of claim 28, configured to be applicable to cell therapy, using a hydrogel produced through a physical host-guest reaction between a hyaluronic acid-cucurbituril conjugate, in which cucurbituril [6] is bonded to hyaluronic acid substituted with cystamine, and/or a Ce6-hyaluronic acid-cucurbituril conjugate, in which Ce6 as a photosensitizer is additionally bonded to the hyaluronic acid-cucurbituril conjugate, and a hyaluronic acid-upconversion nanoparticle conjugate.
